(19) United States
(12) Patent Application Publication
(10) Pub. No.: US 2024/0075179 A1
(43) Pub. Date: Mar. 7, 2024

(54) PHARMACEUTICAL UNIT AND METHOD FOR OPERATING A PHARMACEUTICAL UNIT

(71) Applicant: Syntegon Technology GmbH, Waiblingen (DE)

(72) Inventors: Albrecht Kühnle, Crailsheim (DE); Benjamin Hoffmann, Vellberg (DE); Thomas Kosian, Insingen (DE)

(21) Appl. No.: 18/261,766

(22) PCT Filed: Dec. 20, 2021
(86) PCT No.: PCT/EP2021/086810

§ 371 Date: Jul. 17, 2023

(30) Foreign Application Priority Data
Jan. 22, 2021 (DE) 10 2021 101 403.4

(51) Int. Cl. A61L 9/04; A61L 2/20; A61L 2/26; F24F 3/167

(52) U.S. Cl. CPC A61L 9/046; A61L 2/208; A61L 2/26; F24F 3/167; A61L 2202/25; A61L 2209/14; A61L 2209/211

(57) ABSTRACT

The invention relates to a pharmaceutical unit (12) and to a method for operating a pharmaceutical unit (12).

### BACKGROUND

The invention relates to a pharmaceutical unit, in particular a clean room, and a method for operating a pharmaceutical unit.

Pharmaceutical production facilities are usually designed in the form of barrier systems that are sealed off from their environment.

In pharmaceutical production facilities with high dust and/or aerosol loads, especially if these loads are also classified as highly potent, so-called BIBO (bag-in/bag-out) filter systems are used in the recirculated or exhausted air of the production areas. These are specially designed for low-contamination replacement of the loaded filter elements when the maximum filter load is reached or at the end of a defined replacement interval.

Alternatively, cleanable filter systems are also used, which do not require a filter change. However, these do not allow sufficiently complete cleaning of the filter medium.

Pharmaceutical units must be decontaminated on a regular basis. For this purpose, decontaminant is introduced into the pharmaceutical unit or barrier system so that all contaminated elements come into contact with the decontaminant and are thus decontaminated.

If a decontaminant is used that is harmless to the environment, it is referred to as biodecontamination. Hydrogen peroxide (H2O2) is usually used for this purpose, as it can be degraded to water and oxygen.

Studies have shown that loading of the filter systems can have an impact on the effectiveness of the (bio)decontamination of a barrier system. Particularly in the case of barrier systems used in powder-processing production units, high dust concentrations occur in the recirculated air with correspondingly high loading of the recirculating air filters. Recirculated air refers to air from the barrier system that is recirculated.

As a rule, hygroscopic dusts are also involved, which have the effect that a large proportion of the gaseous decontaminant in the recirculated air is bound in these dusts during (bio)decontamination. If the barrier system is operated in recirculation, the concentration of decontaminant bound in the dusts is no longer available for the decontamination of the entire system.

In practice, the validation and qualification of such units are carried out with new (unloaded) filters, so that this effect only occurs during operation after several hours of operation. There is therefore a risk that such units are operated, quasi unnoticed, in a non-compliant manner.

### SUMMARY

It is the task of the present invention to provide a pharmaceutical unit and a method of operating a pharmaceutical unit, while eliminating the above disadvantages.

This task is solved by a pharmaceutical unit, in particular a clean room, wherein the pharmaceutical unit comprises:

A working space sealed off from its environment and a filter system with a first flow path and a first filter.

The first filter is arranged in the first flow path. The first filter is designed to filter air from the working space. Thus, the air from the working space, which is directed via the first flow path, is filtered by means of the first filter.

The filter system comprises a second flow path and a switching device. The switching device is designed to direct air from the working space via the first flow path and thus via the first filter during an operating phase of the pharmaceutical unit.

The switching device is further designed to direct air from the working space exclusively via the second flow path during a decontamination phase of the pharmaceutical unit. Thus, the first filter is not exposed to decontaminant during the decontamination phase. The first filter is not exposed to air from the working space during the documentation phase.

The first and second flow paths may overlap in at least one region. The first and second flow paths may run together within at least one section, for example within the same conduit. The first and second flow paths may be fluidically coupled to each other in at least one section. However, if the first and second flow paths run together, or overlap in a section, the first filter is located outside of the common section and is only exposed to air from the working space when air is directed through the first flow path.

In the present case, “directed exclusively via the first flow path” means that air from the working space only flows through the first flow path completely along its entire length. For example, air can also flow through common sections of the first flow path and the second flow path. In any case, air from the working space does not flow completely through the second flow path (along its entire length), if we are talking about an exclusive flow through the first flow path, but at most along its sections that may be common to the first flow path.

Similarly, “directed exclusively via the second flow path” means that only the second flow path has air flowing through it completely (along its entire length) from the working space. For example, air can also flow through common sections of the second flow path and the first flow path. In any case, air from the working space does not flow completely through the first flow path (along its entire length) when the second flow path is referred to as the only flow path, but at most along its sections that may be common to the second flow path.

In the present case, directing air over the first flow path or flowing through the first flow path means that the air flows through the first flow path completely, i.e. along its entire length.

Similarly, when air is directed over the second flow path or flows through the second flow path, it is meant that the air flows completely through the second flow path. Decontamination can be carried out by introducing decontaminant into the working space. The decontaminant can be gaseous. The decontamination can be biodecontamination. In particular, the decontaminant may be hydrogen peroxide (H2O2). Similarly, the decontaminant may be a mixture of hydrogen peroxide and air. Conceivably, the decontaminant may also be a mixture of hydrogen peroxide and at least one gas.

In this way, the decontaminant is rerouted at the first filter. Thus, no decontaminant enters or passes through the first filter, which may be loaded. In other words, the first filter is bypassed. In other words, decontaminant and/or air does not flow through the first flow path during the decontamination or decontamination phase.

Thus, especially in aseptic powder applications, the decontaminant is not filtered out of the decontamination process by the first filter (which may be loaded) and is fully available to the decontamination process during the decontamination phase.

Filtering out means that the decontaminant is bound or retained by the first filter or its loading (chemical or physical) and is no longer available for the decontamination process.

The air can be recirculated air from the working space, but also exhausted air from the working space. Recirculated air means air that remains in the working space (is recirculated). Exhausted air means air that is discharged from the working space. Likewise, it is conceivable that the air is outside air introduced into the working space and/or processed (e.g., prefiltered) outside air. Of course, another gas or gas mixture can also be used instead of air.

The filter system may include a second filter configured to filter air from the working space. The second filter can be arranged in the second flow path, so that air from the working space, which is directed via the second flow path, is filtered by means of the second filter.

This means that the first filter, which may be loaded, can be bypassed by a second, unloaded filter during the (bio)decontamination phase. This ensures that the (bio) decontaminant is available for the decontamination process with maximum reproducible effectiveness even after recirculation has taken place.

The switching device can comprise at least one valve. The valve can be designed to fluidically couple or decouple the first flow path and the working space. Alternatively or additionally, the valve may be configured to fluidically couple or decouple the second flow path with the working space. In particular, the switching device may comprise two valves, which may be configured such that a first valve can fluidically couple or decouple the first flow path with the working space. The second valve can fluidically couple or decouple the second flow path with the working space.

Fluidic coupling means that a fluid (gas or liquid) can flow freely between at least two fluidically coupled elements. Accordingly, fluidic decoupling means that a fluid (gas or liquid) cannot flow between two fluidically decoupled elements.

The first filter can be arranged within the pharmaceutical unit, in particular within the working space. Alternatively or additionally, the second filter can be arranged within the pharmaceutical unit, in particular within the working space. In particular, the entire filter system with all components may be arranged within the pharmaceutical unit, in particular within the working space.

However, it is also conceivable that the first filter can be arranged outside the working space, in particular outside the pharmaceutical unit. Alternatively or additionally, the second filter can be arranged outside the working space, in particular outside the pharmaceutical unit. In particular, the entire filter system with all components may be arranged outside the working space, in particular outside the pharmaceutical unit. The filter system or individual components of the filter system can be fluidically coupled, in particular by means of conduits, to the pharmaceutical unit, in particular to the working space.

The filter system can be designed in such a way that, during the operating phase of the pharmaceutical unit, air from the working space is directed via the first filter and the second filter. This allows the second filter to take over the function of the first filter during the operating phase.

The first filter and the second filter can be arranged fluidically in series to each other. The second filter can be connected downstream of the first filter. In particular, the second filter can be a police filter. Thus, the second filter can perform an additional filtering function as well as a backup function. Should the first filter fail, the second filter can take over the filtering function of the first filter so that proper filtration can be maintained. In doing so, the second flow path can direct air from the working space around the first filter and through the second filter in parallel.

The first filter and the second filter can be arranged fluidically parallel to each other. Thus, the air can be directed via the first flow path (e.g. during the operating phase) and thus via the first filter or via the second flow path (e.g. during the decontamination phase) and thus via the second filter.

The switching device can be designed to direct air from the working space exclusively via the first flow path during the operating phase of the pharmaceutical unit. In other words, no air from the working space is directed via the second flow path and thus via the second filter during the operating phase of the pharmaceutical unit. In this way, the load (contamination) of the second flow path or the second filter can be prevented or at least minimized.

Further, the task to be solved is solved by a method for operating a pharmaceutical unit with a working space sealed off from its environment wherein the pharmaceutical unit comprises a filter system with a first flow path and a first filter.

In this regard, the first filter is disposed in the first flow path and is configured to filter air that is directed from the working space via the first flow path.

The filter system comprises a second flow path and a switching device. The switching device can be used to switch the flow of air from the working space between the first flow path and the second flow path.

The method comprises:

Directing air from the working space via the first flow path during an operating phase of the pharmaceutical unit and directing air from the working space exclusively via the second flow path, in particular via the second filter, during a decontamination phase of the pharmaceutical unit.

Decontamination can be carried out by introducing decontaminant into the working space. The decontaminant can be gaseous. The decontamination can be biodecontamination. In particular, the decontaminant may be hydrogen peroxide (H2O2). Similarly, the decontaminant may be a mixture of hydrogen peroxide and air. Conceivably, the decontaminant may also be a mixture of hydrogen peroxide and at least one gas.

The operation of the pharmaceutical unit includes both the operation phase of the pharmaceutical unit and the decontamination phase of the pharmaceutical unit.

During the operating phase of the pharmaceutical unit, the air from the working space, can be directed exclusively via the first flow path and thus the first filter, and can thus be filtered exclusively by means of the first filter.

During the operating phase of the pharmaceutical unit, the air from the working space can be directed via the first flow path and the first filter and additionally via the second flow path and via the second filter. Thus, during the operating phase of the pharmaceutical unit, the air from the working space can be filtered by means of the first filter and additionally by means of the second filter. The second filter is arranged downstream of the first filter in the direction of flow. Accordingly, the second filter is only exposed to air already filtered by the first filter when the air is directed via the first flow path.

To execute the method, a pharmaceutical unit a pharmaceutical unit, in particular a clean room, with features described above can be used.

### BRIEF DESCRIPTION OF THE DRAWINGS

Further features, details and advantages of the invention are apparent from the disclosure and from the following description of embodiments based on the drawings. Showing:

FIG. 1 a schematic illustration of a pharmaceutical unit during an operating phase;

FIG. 2 a schematic illustration of the pharmaceutical unit according to FIG. 1 during a decontamination phase;

FIG. 3 a schematic illustration of a further embodiment of the pharmaceutical unit;

FIG. 4 a schematic illustration of the pharmaceutical unit according to FIG. 3 during the operating phase;

FIG. 5 a schematic illustration of the pharmaceutical unit according to FIG. 3 during the decontamination phase and

FIG. 6 a schematic illustration of a further embodiment of the pharmaceutical unit.

### DETAILED DESCRIPTION

In the following description and in the figures, the corresponding components and elements have the same reference signs. For the sake of better overview, not all reference signs are reproduced in all figures.

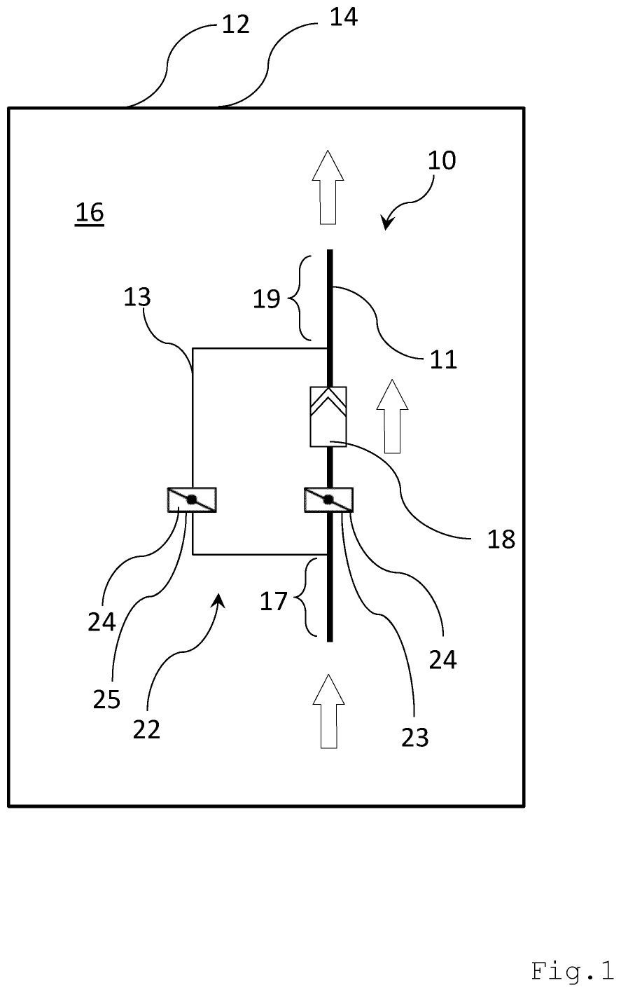

FIG. 1 shows a schematic illustration of a pharmaceutical unit 12 during an operating phase. In the present case, the pharmaceutical unit 12 is designed in the form of a clean room 14. The clean room 14 has a working space 16. This is closed off from its environment.

The clean room 14 further comprises a filter system 10. Presently, the filter system 10 is arranged within the working space 16. That the filter system 10 comprises a first flow path 11 and a first filter 18.

The filter system 10 also has a second flow path 13 and a switching device 22. The switching device 22 is designed in such a way that air from the working space 16 can optionally be directed via the first flow path 11 and thus via the first filter 18, or via the second flow path 13. For this purpose, the switchover device 22 comprises two valves 24. In the illustrated embodiment, a first valve 23 is arranged within the first flow path 11 and a second valve 25 is arranged within the second flow path 13.

By opening/closing the first valve 23, the first flow path 11 can be opened/closed. In other words, by means of the first valve 23, the first flow path 11 can be fluidically coupled or decoupled with the working space 16.

Accordingly, the second flow path 13 can be opened or closed by opening/closing the second valve 25. In other words, by means of the second valve 25, the second flow path 13 can be fluidically coupled or decoupled with the working space 16.

FIG. 1 shows the clean room 14 in the operating phase. Air is sucked from the working space 16 into the filter system 10 and directed via the first flow path 11 and thus the first filter 18. In FIG. 1, the first flow path 11 is highlighted thickly and the path of the air, via the first flow path 11 is indicated with three thick arrows.

In the illustrated state (operating phase), the first valve 23 is open so that air can flow from the working space 16 via the first flow path 11 and thus via the first filter 18. In other words, the first flow path 11 is fluidically coupled to the working space 16 by means of the opened first valve 23.

In the illustrated state (operating phase), the second valve 25 is closed so that air from the working space 16 cannot flow via the second flow path 13. In other words, the second flow path 13 is fluidically decoupled from the working space 16 by means of the closed second valve 25.

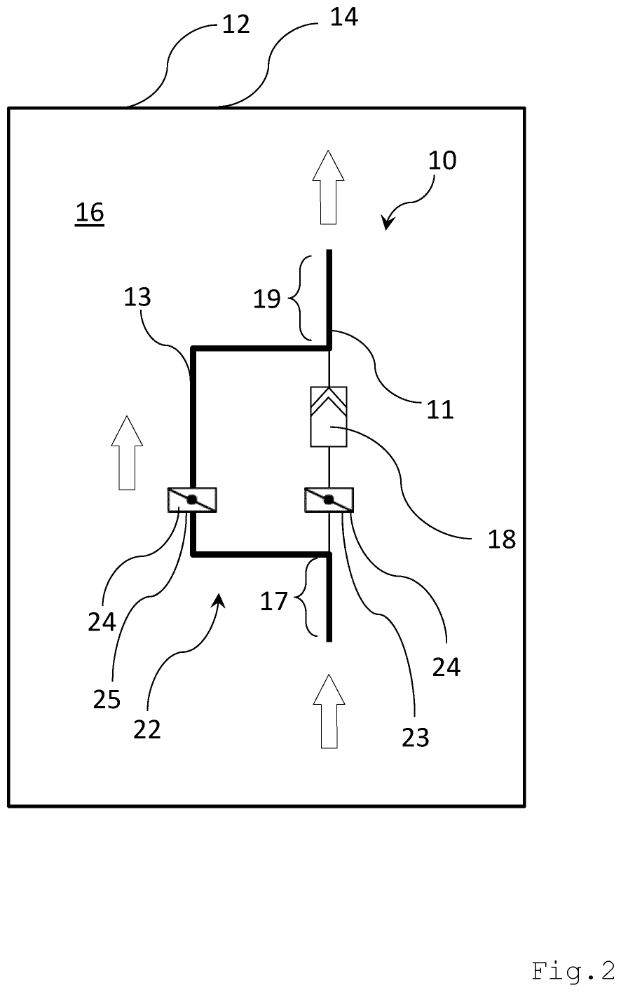

FIG. 2 shows a schematic illustration of the pharmaceutical unit according to FIG. 1 during a decontamination phase. Here, the second flow path 13 is highlighted by a thick line.

During the decontamination phase, the pharmaceutical unit, in particular the working space 16, is decontaminated.

During the decontamination phase, a decontaminant (in particular gaseous hydrogen peroxide) is introduced into the working space 16. Alternatively or additionally, the decontaminant can be introduced into the filter system 10, in particular into the second flow path 13.

The air from the working space 16 and in particular the decontaminant are drawn into the filter system 10 and directed via the second flow path 13. The path of the air or decontaminant via the second flow path 13 is indicated in FIG. 2 by three thick arrows.

In the illustrated state (decontamination phase), the second valve 25 is open so that air can flow from the working space 16 via the second flow path 13. In other words, the second flow path 13 is fluidically coupled to the working space 16 by means of the open second valve 25.

In the illustrated state (decontamination phase), the first valve 23 is closed so that air from the working space 16 cannot flow via the first flow path 11 and thus via the first filter 18. In other words, the first flow path 11 and thus the first filter 18 are fluidically decoupled from the working space 16 by means of the closed first valve 23.

In other words, during the decontamination phase, air or decontaminant does not flow through the first filter 18. The loading of the first filter 18 or the particles filtered out in the filter 18 during the operating phase can thus not interact with the air or the decontaminant. The decontaminant is thus fully available for the decontamination process during the decontamination phase.

In the embodiments shown in FIGS. 1 to 6, the first flow path 11 and the second flow path 13 have a first common section 17 and a second common section 19. In the present case, the first flow path 11 and the second flow path 13 extend along a common conduit in the two common sections 17, 19. In other words, the first flow path 11 and the second flow path 13 are fluidically coupled along the common sections 17, 19. Of course, it is conceivable that the first flow path 11 and the second flow path 13 do not have a common section and are formed separately from each other.

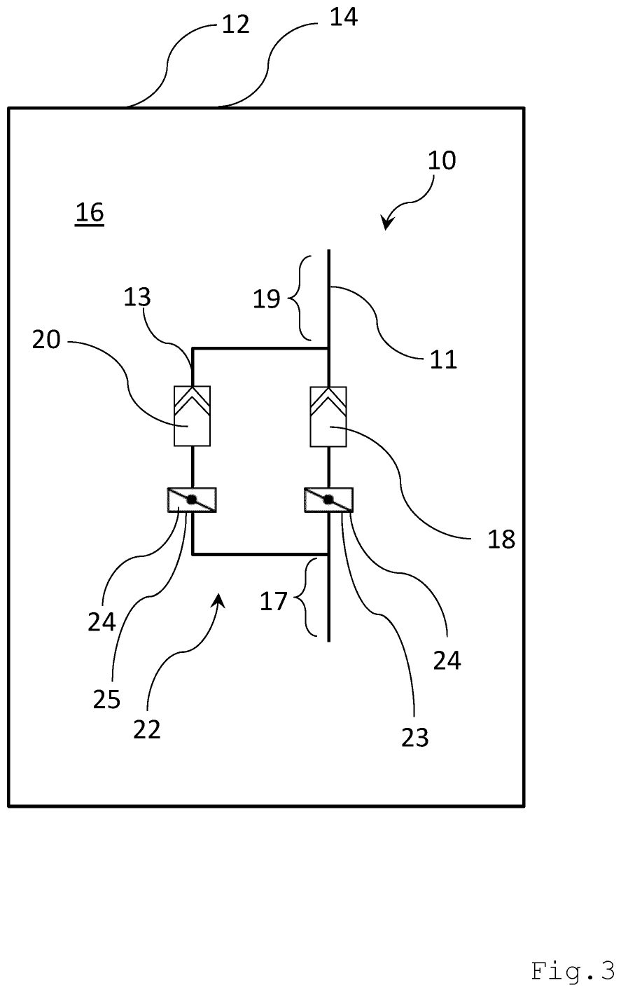

FIG. 3 shows a schematic illustration of a further embodiment of the pharmaceutical unit 12. The embodiment shown differs from the preceding embodiment in that a second filter 20 is arranged within the second flow path 13. When air from the working space 16 is directed through the second flow path 13, the air also flows through the second filter 20.

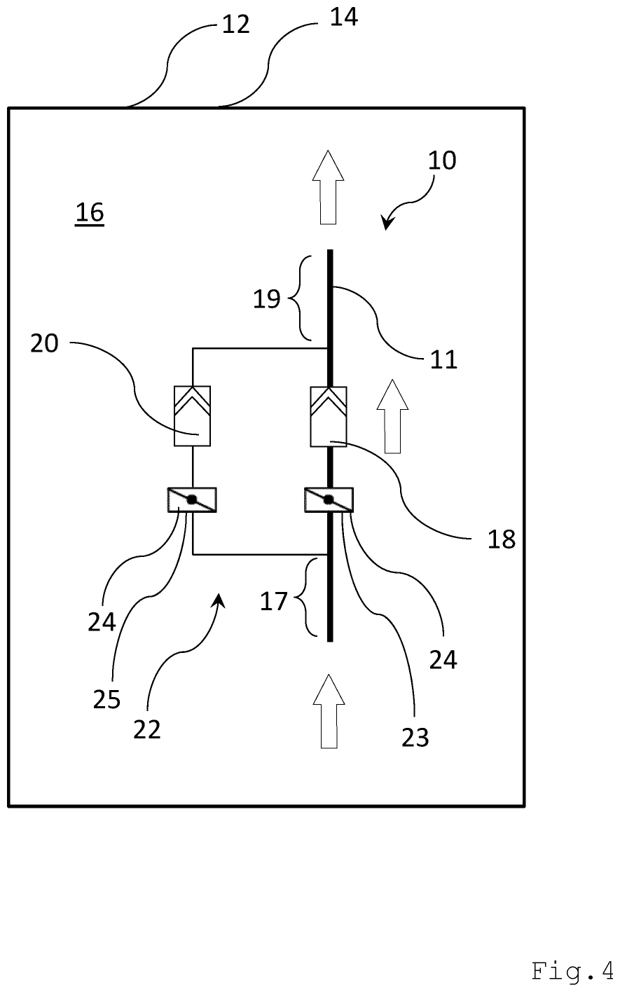

FIG. 4 shows a schematic illustration of the pharmaceutical unit 12 according to FIG. 3 during the operating phase.

During the operating phase, the air is drawn from the working space 16 into the filter system 10 and directed via the first flow path 11 and thus via the first filter 18. For this purpose, the first flow path 11 is fluidically coupled to the working space 16 by means of the opened first valve 23.

In the present case, during the operating phase, the second flow path 13 and thus the second filter 20 are fluidically decoupled from the working space 16 by means of a closed second valve 25. In other words, during the operating phase, no air flows over the second flow path 13 or the second filter 20. In this way, it can be prevented that the second flow path 13 and in particular the second filter 20 is loaded during the operating phase, for example by particles generated during the operating phase.

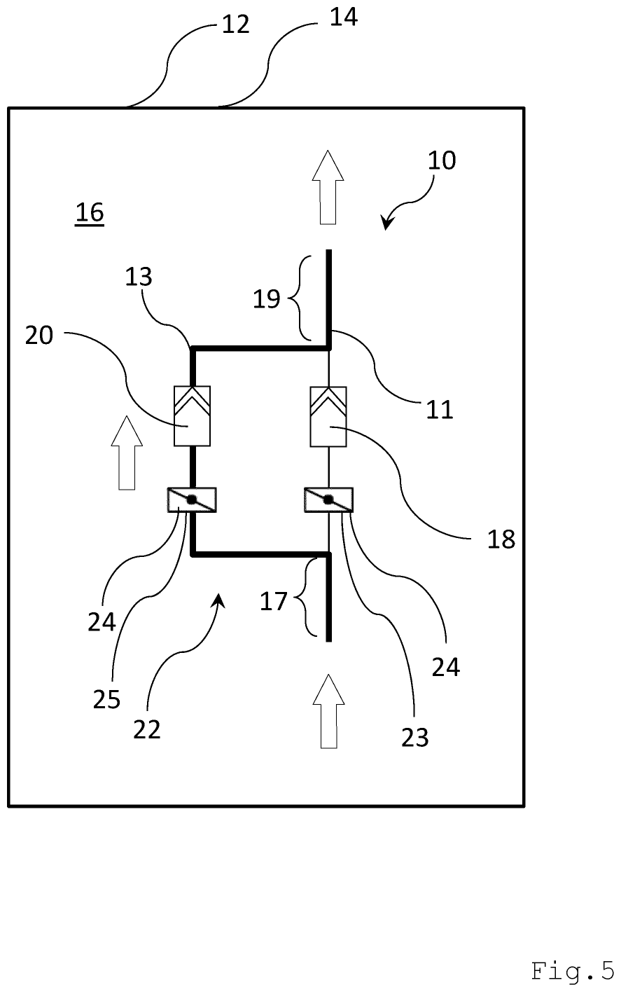

FIG. 5 shows a schematic illustration of the pharmaceutical unit 12 according to FIG. 3 during the decontamination phase.

During the decontamination phase or decontamination, the air is drawn from the working space 16 into the filter system 10 and directed via the second flow path 13 and thus via the second filter 20. The path of the air through the second flow path 13 is indicated in FIG. 5 by three thick arrows.

During the decontamination phase, the first flow path 11 is fluidically decoupled from the working space 16 by means of the closed first valve 23.

During the decontamination phase, the second flow path 13 and thus also the second filter 20 are fluidically coupled to the working space 16 by means of the open second valve 25.

Thus, the decontaminant is directed only via the second flow path 13 and thus only via the second filter 20. The decontaminant is not directed via the first flow path 11 and thus does not enter the first filter 18. The decontaminant can thus not interact with the loading of the first filter 18, so that the decontaminant cannot be filtered out (see above) and is fully available for the decontamination process during the decontamination phase.

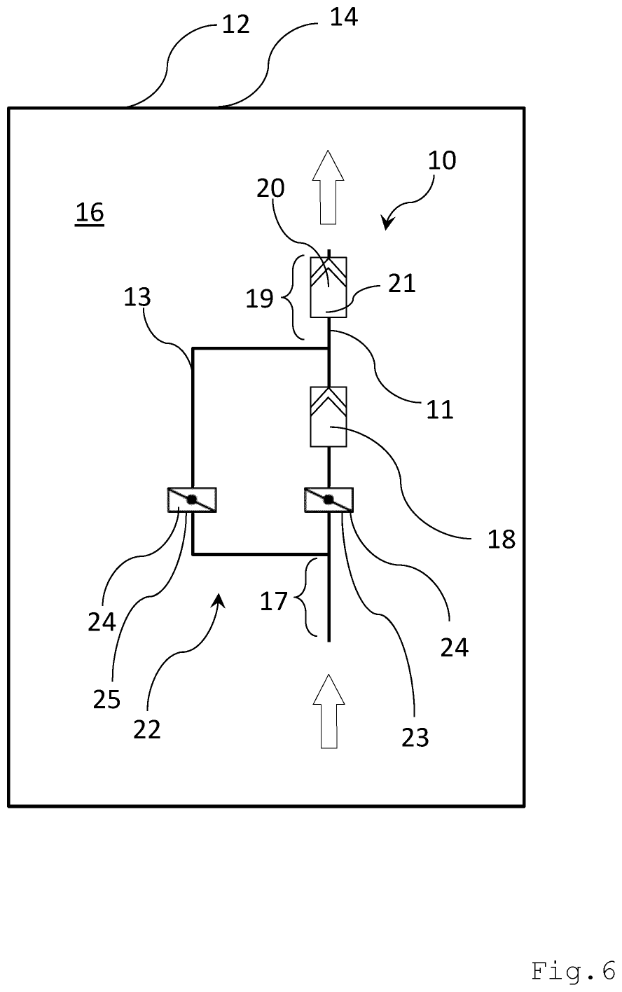

FIG. 6 shows a schematic illustration of a further embodiment of the pharmaceutical unit 12. Here, the first filter 18 and the second filter 21 are fluidically connected in series.

Here, the first filter 18 is arranged within the first flow path 11. The second filter 21 is arranged downstream of the first filter 18 and within the common section 19 of the first flow path 11 and the second flow path 13.

In the arrangement shown, the second filter 20 fulfills the function of a so-called police filter 21. In the operating phase, the air is drawn from the working space 16 into the filter system 10 and directed through the first flow path 11.

For this purpose, the first valve 23 is open so that the first flow path 11 is fluidically coupled to the working space 16. The second valve 25 is closed so that the second flow path 13 is fluidically decoupled from the working space 16.

The air which is directed through the first flow path 11 is filtered by means of the first filter 18. The air filtered by means of the first filter 18 is further directed through the common section 19 and through the second filter 20. The second filter 20 fulfills a backup function. If the first filter 18 fails during the operating phase, the second filter 20 (as a police filter 21) can take over the function of the first filter 18 and thus ensure the filtering function of the filter system 10.

For the decontamination phase, the first valve 23 is closed and the second valve 25 is opened. This directs the air or decontaminant drawn into the filter system 10 from the working space 16 via the second flow path 13 so that the first filter 18 is bypassed.

In the present case, the second flow path 13 leads into the common section 19 upstream of the second filter 20, thus bypassing the first filter 18 during the decontamination phase and directing the air or decontaminant exclusively through the second filter 20.

In other words, during the decontamination phase, the air or decontaminant is rerouted around the first filter 18 by means of the second flow path 13. The first valve 23, shown in FIG. 6 and closed during the decontamination phase, prevents the air or decontaminant from flowing over the first filter 18. In other words, the closed first valve 23 fluidically decouples the first filter 18 from the working space 16 during the decontamination process.

During the decontamination process, the decontaminant or the air from the working space 16 is directed exclusively via the second filter 20. The decontaminant therefore does not come into contact with the first filter 18 or its load.

1. A pharmaceutical unit (12), comprising:
a working space (16) that is sealed off from its environment,
a filter system (10) comprising a first flow path (11) and a first filter (18), wherein the first filter (18) is disposed in the first flow path (11) and is configured to filter air that is directed from the working space (16) via the first flow path (11), wherein the filter system (10) comprises a second flow path (13) and a switching device (22), wherein the switching device is configured to direct air from the working space (16) via the first flow path (11) and thus via the first filter (18) during an operating phase of the pharmaceutical unit (12) and to direct the air from the working space (16) exclusively via the second flow path (13) during a decontamination phase of the pharmaceutical unit (12), so that the first filter (18) is not exposed to a decontaminant during the decontamination phase in which the decontaminant is supplied to the working space (16), wherein the air is recirculating air.

2. The pharmaceutical unit (12) according to claim 1, wherein the decontamination phase is a biodecontamination phase in which hydrogen peroxide is introduced into the working space (16).

3. The pharmaceutical unit (12) according to claim 1, wherein the filter system (10) comprises a second filter (20) arranged in the second flow path (13) and configured to filter air from the working space (16) that is directed via the second flow path (13).

4. The pharmaceutical unit (12) according to claim 1, wherein the switching device (22) comprises at least one valve (24, 23, 25) the at least one valve (24, 23, 25) being configured to fluidically couple and/or decouple the first flow path (11) and/or the second flow path (13) to the working space (16).

5. The pharmaceutical unit (12) according to claim 3, wherein the first filter (18) and/or the second filter (20) is arranged within the pharmaceutical unit (12).

6. The pharmaceutical unit (12) according to claim 3, wherein the first filter (18) and/or the second filter (20) is arranged outside the working space (16).

7. The pharmaceutical unit (12) according claim 3, wherein the switching device is configured to direct air from the working space (16), via the first filter (18) and the second filter (20) during the operating phase of the pharmaceutical unit (12).

8. The pharmaceutical unit (12) according to claim 3, wherein the first filter (18) and the second filter (20) are fluidically arranged in series with each other, wherein the second filter (20) is arranged downstream of the first filter (18), wherein the second flow path (13) directs the air from the working space (16) in parallel around the first filter (18) and through the second filter (20).

9. The pharmaceutical unit (12) according to claim 3, wherein the first filter (18) and the second filter (20) are arranged fluidically parallel to each other.

10. A method of operating a pharmaceutical unit (12) comprising a working space (16) that is sealed off from its environment, wherein the pharmaceutical unit (12) comprises a filter system (10) having a first flow path (11) and a first filter (18), wherein the first filter (18) is disposed in the first flow path (11) and is configured to filter air that is directed from the working space (16) via the first flow path (11), wherein the filter system (10) comprises a second flow path (13) and a switching device (22) configured such that a flow of air from the working space (16) can be switched between the first flow path (11) and the second flow path (13), wherein the method comprises:
Directing air from the working space (16) via the first flow path (11) in an operating phase of the pharmaceutical unit (12) and directing air from the working space (16) exclusively via the second flow path (13) during a decontamination phase of the working space (16), wherein the air is recirculating air.

11. The method according to claim 10, wherein during the operating phase of the pharmaceutical unit (12), air from the working space (16) is additionally conducted via the second flow path (13).

12. (canceled)

13. The pharmaceutical unit (12) according to claim 1, wherein the pharmaceutical unit (12) is a clean room (14).

14. The pharmaceutical unit (12) according to claim 1, wherein the decontaminant is air from the working space (16).

15. The pharmaceutical unit (12) according to claim 1, wherein the decontaminant is a gaseous contaminant.

16. The pharmaceutical unit (12) according to claim 4, wherein the at least one valve (24, 23, 25) includes two valves (24, 23, 25).

17. The pharmaceutical unit (12) according to claim 8, wherein the second filter (20) is a police filter (21).

18. The method according to claim 10, wherein during the decontamination phase a decontaminant is introduced into the working space (16).

19. The method according to claim 18, wherein the decontaminant is a gaseous decontaminant.

20. The method according to claim 18, wherein the decontaminant is hydrogen peroxide.
